(19) United States
(12) Patent Application Publication
(10) Pub. No.: US 2016/0271097 A1
(43) Pub. Date: Sep. 22, 2016

(54) FORMULATION OF OLOPATADINE

(71) Applicant: NEPHRON PHARMACEUTICALS CORPORATION, Orlando, FL (US)

(72) Inventors: Eric Primelles-Perez, Orlando, FL (US); Jessica R. Sims, Orlando, FL (US)

(21) Appl. No.: 15/033,171

(22) PCT Filed: Aug. 27, 2014
(86) PCT No.: PCT/US2014/052888

§ 371 Date: Apr. 29, 2016

Related U.S. Application Data
(60) Provisional application No. 61/898,347, filed on Oct. 31, 2013.

(51) Int. Cl. A61K 31/335

(52) U.S. Cl. CPC A61K 31/335

(57) ABSTRACT

Stable formulations of Olopatadine, methods of making such formulations and methods of treatment using such formulations are provided.

### FIELD OF THE INVENTION

The invention relates to stable formulations of carboxylic acid derivatives of doxepin, methods of making such formulations and methods of treatment.

### BACKGROUND

The prior art has identified challenges relating to preparing and preserving stable formulations of olopatadine. In the case of ophthalmic formulations, additional challenges come into play, including solubility and viscosity.

U.S. Pat. Nos. 4,871,865 and 4,923,892 teach carboxylic acid derivatives of doxepin, including olopatadine (chemical name: 11-[(Z)-3-(Dimethylamino)propylidene]-6-11-dihydrodibenz[b,e]oxepin-2-acetic acid). These patents teach various formulations, including ophthalmic formulations.

U.S. Pat. No. 5,116,863 teaches that carboxylic acid derivatives of doxepin, in particular, olopatadine, have anti-allergic and anti-inflammatory activity. The described formulations include a wide range of acceptable carriers; however, only oral and injection administration forms are mentioned.

U.S. Pat. No. 5,641,805 teaches ophthalmic formulations of olopatadine for treating allergic eye diseases. According to the '805 patent, the topical formulations may be solutions, suspensions or gels. The formulations contain olopatadine, an isotonicity agent, and “if required, a preservative, a buffering agent, a stabilizer, a viscous vehicle and the like.” [See Col. 6, lines 30-43.] “[P]olyvinyl alcohol, polyvinylpyrrolidone, olyacrylic acid or the like” are mentioned as viscous vehicles. [See Col. 6, lines 55-57.]

U.S. Pat. No. 6,375,973 teaches ophthalmic formulations of olopatadine. The formulations include a polymeric quaternary ammonium compound as a preservative, provided that the composition does not contain benzalkonium chloride. The compositions may also include viscosity modifying agents such as: cellulosic ethers, such as, hydroxypropyl methyl cellulose (HPMC), hydroxyethyl cellulose (HEC), ethyl hydroxyethyl cellulose, hydroxypropyl cellulose, methyl cellulose, and carboxymethyl cellulose; carbomers (e.g. Carbopol®; polyvinyl alcohol; polyvinyl pyrrolidone; alginates; carrageenans; and guar, karaya, agarose, locust bean, and xanthan gums.

U.S. Pat. No. 6,743,439 teaches formulations of cationic drugs (including olopatadine) and cationic preservatives, together with a sulfonated styrene/maleic anhydride copolymer. According to the '439 patent, solutions that contain water-soluble polystyrene sulfonic acid to enhance the solubility of a drug can be difficult to preserve because the negatively charged polystyrene sulfonic acid interacts with the cationic preservative, reducing the preservative's ability to function as a preservative. The ‘439 patent suggests that solutions containing a sulfonated styrene/maleic anhydride copolymer are easier to preserve than similar solutions containing polystyrene sulfonic acid.

U.S. Pat. Nos. 6,995,186 and 7,402,609 teach that polyvinylpryrrolidone and polystyrene sulfonic acid, unlike polyvinyl alcohol and the polyacrylic acid carbomer 974P, enhance the physical stability of solutions containing 0.2-0.6% olopatadine. Solutions are prepared with a pH from 6.5-7.5 and a viscosity of 1-2 cps, and consist essentially of: a) 0.18-0.22% (w/v) olopatadine; b) 1.5-2% (w/v) polyvinylpyrrolidone having an average molecular weight of 50,000-60,000; c) a preservative selected from benzalkonium chloride; benzododecinum bromide; and polyquaternium-1; d) edetate disodium; e) a tonicity-adjusting agent selected from the group consisting of mannitol and sodium chloride; f) a buffering agent selected from phosphates and borates; g) optionally a pH-adjusting agent selected from NaOH and HCl; and h) water. U.S. Pat. No. 8,399,508 teaches a solution that does not contain polymeric ingredients for enhancing the solubility of olopatadine or the physical stability of the solution. The solutions “do not contain polyvinylpyrrolidone, polystyrene sulfonic acid, polyvinyl alcohol, polyvinyl acrylic acid, hydroxypropylmethyl cellulose, sodium carboxymethyl cellulose or xanthan gum”. Instead, the solutions rely on a very low pH to stabilize the solutions (e.g., “pH-adjusting agents in an amount sufficient to cause the composition to have a pH of 3.6-3.8.” As such, they are not well suited for the eye.

### SUMMARY OF THE INVENTION

It would be desirable to have a stable ophthalmic solution of olopatadine that (i) delivers effective amounts of olopatadine, (ii) avoids the need for a preservative, (iii) avoids the need for unacceptably low pH levels, (iv) is simple to manufacture and (v) is stable. It was discovered, unexpectedly and contrary to the teachings of the prior art, that stable, ophthalmic, formulations of olopatadine can be prepared with only olopatadine and polyvinyl alcohol. There is no need for any other polymeric component. There is no need for a preservative. There is no need for any additional substance to achieve necessary solubility or viscosity. The formulation is stable, even at neutral pH.

According to one aspect of the invention, a pharmaceutical composition is provided. The composition is an aqueous solution containing 11- (3 dimethylaminopropylidene)-6,11-dihydrodibenz(b,e) oxepin-2-acetic acid or a pharmaceutically acceptable salt thereof, and polyvinyl alcohol at a concentration of greater than 0.50% w/w and less than 1.75% w/w, wherein the pH is between 5.0 and 8.0, optionally between 6.8 and 7.2, and the osmolality is between 260 and 340 mOsm/kg. In any embodiment, the 11-3-(Dimethylamino)propylidene]-6-11-dihydrodibenz[b,e]oxepin-2-acetic acid can be 11-([Z]-3 dimethylaminopropylidene)-6- 11 dihydrodibenz(b,e) oxepin-2-acetic acid. In any embodiment, the 11-(3 dimethylaminopropylidene)-6,11-dihydrodibenz(b,e) oxepin-2-acetic acid can be present in an amount between 0.5mg/mL and 3.0 mg/mL. In any embodiments, the 11-(3 dimethylaminopropylidene)-6,11-dihydrodibenz(b,e) oxepin-2-acetic acid can be present in an amount between 1.5 mg/mL and 2.5 mg/mL. The pharmaceutical composition, in any of the foregoing embodiments, may further contain a chelating agent. The chelating agent, for example, may be ethylenediaminetetraacetate (EDTA). Other chelating agents are described below. The pharmaceutical composition, in any of the foregoing embodiments, may further contain a buffer. The buffer, for example, can be disodium phosphate and sodium chloride.

Other buffers are described below.

The pharmaceutical composition, in any of the embodiments, may contain the polyvinyl alcohol at a concentration of between 0.50 and 1.75% w/w. The pharmaceutical composition, in any of the embodiments, may contain the polyvinyl alcohol at a concentration of between 0.60 and 1.50% w/w or even 0.75 and 1.35% w/w.

According to another aspect of the invention, a pharmaceutical composition is provided. The composition is an aqueous solution containing 11-[(Z)-3-(Dimethylamino)propylidene]-6-11-dihydrodibenz[b,e]oxepin-2-acetic acid present in an amount between 1.5 mg/mL and 2.5 mg/mL, polyvinyl alcohol at a concentration of between 0.50 and 1.75% w/w, disodium phosphate, sodium chloride, and EDTA, and wherein the pH of the composition is between 5.0 and 8.0, preferably between 6.8 and 7.2, and the osmolality is between 260 and 340 mOsm/kg.

The pharmaceutical composition, in any of the foregoing embodiments, may be free of benzalkonium chloride. The pharmaceutical composition, in any of the embodiments, may be free of polymeric quaternary ammonium compounds that are preservatives. The pharmaceutical composition, in any of the embodiments, may be free of any preservative other than a chelating agent. The pharmaceutical composition, in any of the embodiments, may be free of any preservative other than EDTA. The pharmaceutical composition, in any of the embodiments, may be free of any preservative. The pharmaceutical composition, in any of the embodiments, may be free of viscosity enhancing agents other than polyvinyl alcohol. The pharmaceutical composition, in any of the embodiments, may be free of povidone (polyvinylpyrrolidone). The pharmaceutical composition, in any of the embodiments, may be free of polymers other than polyvinyl alcohol. The pharmaceutical composition, in any of the embodiments, may be free of benzalkonium chloride and free of povidone. The pharmaceutical composition, in any of the embodiments, may be free of any preservative and free of povidone. The pharmaceutical composition, in any of the embodiments, may be free of any preservative and free of any polymer other than polyvinyl alcohol.

In one embodiment, the composition consists essentially of, or consists of, an aqueous solution containing 11-[(Z)-3-(Dimethylamino)propylidene]-6-11-dihydrodibenz[b,e]oxepin-2-acetic acid present in an amount between 1.5 mg/mL and 2.5 mg/mL, polyvinyl alcohol at a concentration of between 0.50 and 1.75% w/w, disodium phosphate, sodium chloride, and EDTA, and wherein the pH of the composition is between 5.0 and 8.0, preferably between 6.8 and 7.2, and the osmolality is between 260 and 340 mOsm/kg.

According to another aspect of the invention, a method for treating an allergic condition is provided. The method involves administering topically to the eye any of the pharmaceutical compositions described above.

According to another aspect of the invention, a method of manufacture is provided. The method involves dissolving polyvinyl alcohol into an aqueous solution to form an intermediate solution, and then dissolving 11-[(Z)-3-(Dimethylamino)propylidene]-6-11-dihydrodibenz[b,e]oxepin-2-acetic acid or a pharmaceutically acceptable salt thereof into the intermediate solution to form a final solution, wherein the polyvinyl alcohol dissolved into the aqueous solution and the 11-[(Z)-3-(Dimethylamino)propylidene]-6-11-dihydrodibenz[b,e]oxepin-2-acetic acid dissolved into the intermediate solution are present in amounts such that the final solution contains between 0.50 and 1.75% w/w polyvinyl alcohol of all ingredients in the final solution and between 1.5 mg/mL and 2.5 mg/mL 11-[(Z)-3-(Dimethylamino)propylidene]-6-11-dihydrodibenz[b,e]oxepin-2-acetic acid in the final solution.

### BRIEF DESCRIPTION OF THE FIGURES

FIG. 1 is a graph showing the effect on viscosity of formulations containing different modifiers in different amounts versus a control formulation.

FIG. 2 is a graph showing the effect on viscosity of formulations containing different grades of PVA.

### DETAILED DESCRIPTION

U.S. Pat. Nos. 4,871,865 and 4,923,892 describe carboxylic acid derivatives of doxepin, including 11- (3 dimethylaminopropylidene)-6,11-dihydrodibenz(b,e) oxepin-2-acetic acid and, in particular, olopatadine (chemical name: 11-[(Z)-3-(Dimethylamino)propylidene]-6-11-dihydrodibenz[b,e]oxepin-2-acetic acid). Olopatadine ophthalmic solutions are commercially available at 0.2% mg/mL concentrations and are used to treat eye symptoms of allergic conditions, such as inflammation, itching, watering, and burning. In the present invention, olopatadine solutions typically contain amounts of olopatadine between 0.5 mg/mL and 3.0 mg/mL. In some embodiments the solutions contain between 1.5 mg/mL and 2.5 mg/mL of olopatadine, and in yet other embodiments the solutions contain between 1.8 and 2.3 mg/mL olopatadine.

The olopatadine can be supplied as a pharmaceutically acceptable salt. In one embodiment, the salt is olopatadine hydrochloride. “Pharmaceutically acceptable salt”, in general, refers to those salts which are, at useful concentrations and within the scope of sound medical judgment, suitable for use in contact with the human eye without undue toxicity, irritation, allergic response, and the like. Pharmaceutically acceptable salts are well known in the art. For example, Berge et al., describe pharmaceutically acceptable salts in detail in J. Pharmaceutical Sciences (1977) 66:1-19.

Pharmaceutically acceptable salts of the compounds describe herein include those derived from suitable inorganic and organic acids and bases. Examples of pharmaceutically acceptable, nontoxic acid addition salts are salts formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4alkyl)4 salts. Representative alkali or alkaline earth metal salts include sodium, lithium, potassium, calcium, magnesium, and the like. Further pharmaceutically acceptable salts include, when appropriate, quaternary salts.

Polyvinyl alcohol (PVA) is a water-soluble synthetic polymer. It has the idealized formula [CH₂CH(OH)]ₙ. PVA typically is prepared by first polymerizing vinyl acetate, and the resulting polyvinylacetate is converted to the PVA. Other precursor polymers are sometimes used, with formate, chloroacetate groups instead of acetate. The conversion of the polyesters is usually conducted by base-catalysed transesterification with ethanol:

[CH₂CH(OAc]ₙ+C₂H₅OH→[CH₂CH(OH)]ₙ+C₂H₅OAc

Peroxides are not used during the PVA polymerization, and therefore the peroxide elimination step is not needed during the manufacturing process. The properties of the polymer depend on the molecular weight and amount of residual ester groups. There are three commercially available grades of PVA according to the Handbook of Pharmaceutical Excipients. All of them are USP reference materials, have the same CAS #9002-89-5, and the grades depend on the degree of polymerization n obtained during manufacturing. They are:

| | Molecular | Dynamic viscosity of 4% w/v |
|---|---|---|
| Grade | Weight | aqueous solution at 20° C. (mPa s) |
| High Viscosity | ~200 000 | 40.0-65.0 |
| Medium Viscosity | ~130 000 | 21.0-33.0 |
| Low Viscosity | ~20 000 | 4.0-7.0 |

According to the present invention, when different grades of PVA or their combinations are used at a concentration of between 0.50 and 1.75% w/w total, solution viscosities between 1.09 and 4.74 cPs are obtained. Concentrations of such PVA below 0.50% are insufficient to permit dissolution of olopatadine concentrations of about 2.0 mg/mL. Concentrations of such PVA at 1.80% or above result in undesirable viscosities. Concentrations of such PVA typically employed in the invention are between 0.75 and 1.35% w/w.

The solutions of the invention can be free of povidone. The solutions of the invention can be free of viscosity enhancing agents other than polyvinyl alcohol. The solutions of the invention can be free of polymers other than polyvinyl alcohol.

During manufacture, pH can be adjusted. In embodiments, the pH is typically between 5.0 and 8.0. In any of the embodiments, the pH can be between 6.8 and 7.2. The osmolality of the solutions of the invention are maintained in ranges typically used within the eye. As such, osmolality typically is between 260 and 340 mOsm/kg. The solutions of the invention may contain a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. In some embodiments, the chelating agent is EDTA. The solutions of the invention can be free of the preservative benzalkonium chloride. The solutions can be free of polymeric quaternary ammonium compounds that are preservatives. The solutions can be free of any preservative other than a chelating agent. The solutions can be free of any preservative, including free of chelating agents. Exemplary preservatives include antioxidants, chelating agents, antimicrobial preservatives, antifungal preservatives, alcohol preservatives, and acidic preservatives. Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, sodium sulfite and vitamin E polyethylene glycol succinate. Exemplary antimicrobial preservatives include benzalkonium chloride, benzethonium chloride, benzyl alcohol, boric acid, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, and potassium metabisulfite.

The solutions of the invention can include a buffer. In any of the embodiments, the buffer can be disodium phosphate and sodium chloride. Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

The solutions of the invention can be used to treat a subject with an allergic condition of the eye. “Treat”, “treating” and “treatment” encompass an action that occurs while a subject is suffering from a condition which reduces the severity of the condition (or a symptom associated with the condition) or retards or slows the progression of the condition (or a symptom associated with the condition). This is therapeutic treatment. “Treat”, “treating” and “treatment” also encompasses an action that occurs before a subject begins to suffer from the condition (or a symptom associated with the condition) and which inhibits the onset of or reduces the severity of the condition (or a symptom associated with the condition). This is prophylactic treatment.

Subjects are treated with effective amounts of the solutions of the invention. An “effective amount” of a compound generally refers to an amount sufficient to elicit the desired biological response, i.e., treat the condition. As will be appreciated by those of ordinary skill in this art, the effective amount of a compound described herein may vary depending on such factors as the condition being treated, the mode of administration, and the age and health of the subject. The condition treated by the solutions of the invention can be an allergic condition manifested in the eye, such as inflammation, itching, watering, and burning. An effective amount encompasses therapeutic and prophylactic treatment.

For therapeutic treatment, an effective amount is an amount sufficient to provide a therapeutic benefit in the treatment of a condition or to reduce or eliminate one or more symptoms associated with the condition. This may encompass an amount that improves overall therapy, reduces or avoids symptoms or causes of the condition, or enhances the therapeutic efficacy of another therapeutic agent.

For prophylactic treatment, an effective amount is an amount sufficient to prevent, delay the onset of, or reduce the severity of a condition, or one or more symptoms associated with the condition, or prevent its recurrence. This may encompass an amount that improves overall prophylaxis or enhances the prophylactic efficacy of another prophylactic agent.

A subject as used herein means a human.

Administering as used herein means contacting affected tissue of the subject, for example by topically applying eye drops to the eye.

### EXAMPLES

Summary

It is known that olopatadine will not go into aqueous solution at levels useful for ophthalmic administration for adults (1.5-2.5 mg/mL) without a solubilizing agent. Povidone is used in commercial ophthalmic formulations of olopatadine in order to (i) enhance the solubility of olopatadine, (ii) increase viscosity of the formulations, and (iii) increase stability of the formulations.

Formulations of olopatadine and Povidone were tested, using amounts of povidone at or below the amount present in commercial formulations of olopatadine (1.8%). The % povidone, even when there was treatment to remove potential residual peroxide, had a negative effect on the formulation stabilities increasing the detected levels for the Olopatadine N-Oxide and Carbaldehyde degradation products.

Olopatadine

Name: {(11Z)-11- [3-(dimethylamino)propylidene]-6,11-dihydrodibenzo[b,e]oxepin-2-yl} acetic acid

Molecular Formula: C₂₁H₂₃NO₃

Molecular Weight: 337.42

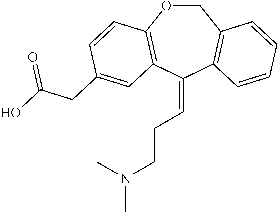 [SMILES: CN(C)CC/C=C1/c2ccccc2COc2ccc(CC(=O)O)cc21]

Olopatadine N-Oxide: Olopatadine Hydrochloride Related Compound B

Name: RS (Z)-3-{2-(Carboxymethyl)dibenzo[b, e]oxepin-11(6H)-ylidene}-N,N-dimethylpropan-1-amine oxide

Molecular Formula: C₂₁H₂₃NO₄

Molecular Weight: 353.42

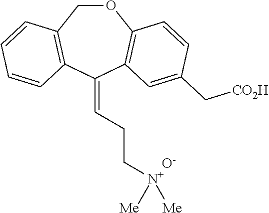

Olopatadine Carbaldehyde:

Name: (11Z)-11-[3-(Dimethylamino)propylidene]-6,11-dihydrodibenz[b,e]oxepin-2-ethanal

Molecular Formula: C₂₁H₂₃NO₂

Molecular Weight: 321.42

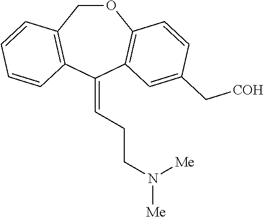

Formulations were prepared using several different modifiers [hydroxypropylmethylcellulose (HPMC), carboxymethylcellulose (CMC) and polyvinyl alcohol (PVA)] to determine whether the amount of povidone could be reduced and replaced with another polymer, while preserving the benefits provided by povidone in terms of solubility, viscosity, and stability. Decreasing amounts of povidone were combined with increasing amounts of modifier. Replacing some of the povidone with amounts of any one of the three modifiers improved the formulation in terms of the two degradants, although degradation still occurred.

The effect of the three modifiers on viscosity was measured. HPMC and CMC, when substituted in amounts at or even less than the amount of povidone present in commercial formulations, increased viscosity to unacceptable levels. PVA, however, did not increase viscosity to unacceptable levels.

Various amounts of PVA were then tested with various amounts of povidone. It was discovered, surprisingly, that the Olopatadine N-Oxide and Carbaldehyde degradation products were completely eliminated when povidone was eliminated and replaced with an equivalent amount of PVA. It also was discovered, surprisingly, that even when the amount of PVA was reduced and no povidone was present, the Olopatadine N-Oxide and the Olopatadine Carbaldehyde were completely absent.

It was decided to run additional tests on formulations of PVA without povidone. It was discovered that when the amount of PVA was reduced and no povidone was present, the formulation had a viscosity within acceptable limits.

The effects of the total amount of PVA for the three grades in the formulations viscosities were evaluated between 0.50% and 1.75%. Additionally, a PVA Mixture Design of Experiment was carried out at two levels (0.75% and 1.35%) to evaluate the effect of the different grades in the formulation viscosities.

On the basis of these experiments, described in greater detail below, it was discovered surprisingly that a combination of olopatadine and PVA was sufficient to produce an ophthalmic formulation having acceptable olopatadine solubility, stability and viscosity.

### Example 1

Commercial formulations of olopatadine were determined to have approximately 1.8 mg/mL Povidone K-30, 2.2 mg/mL olopatadine and a viscosity of about 1.35 cPs (1-2 cPs as per the U.S. Pat. No. 6,995,186). It has been established that povidone raw material may contain peroxides as trace contaminants from the polymerization reaction, which can lead to degradation of an active pharmaceutical ingredient that is sensitive to oxidation.

During these studies three different polymer modifiers were evaluated in order to determine whether povidone could be reduced or replaced. These modifiers were: Hydroxypropyl Methylcellulose, Carboxymethylcellulose, and Polyvinyl Alcohol. Hydroxypropyl Methylcellulose (HPMC) is a polymer used in ophthalmic solutions to increase drug solubility and increase viscosity. Carboxymethylcellulose (CMC) is a polymer used in ophthalmic solutions to increase viscosity. Polyvinyl Alcohol (PVA) is a water-soluble synthetic polymer that has been used in ophthalmic solutions to increase viscosity.

Initial experiments were conducted to examine the possible concentration ranges for the organic modifiers which would initially solubilize Olopatadine during formulation and maintain the solution stability such that no crystallization of the drug product would occur.

Materials: Olopatadine Hydrochloride, DSM; Povidone K-30, Spectrum; Hypromellose 2910, Spectrum; Carboxymethylcellulose Sodium, Spectrum; Polyvinyl Alcohol, Spectrum; Disodium Phosphate, Dibasic, Dihydrate, EMD; Sodium Chloride, J. T. Baker; EDTA, Dihydrate, J. T. Baker

Preparation of Stock Salt Solution: Dissolved 12.538 g of Disodium Phosphate Monohydrate, 12 g Sodium Chloride, and 0.254 g EDTA into a 200 mL volumetric flask, using purified water. Diluted to volume with purified water.

Preparation of Stock Povidone Solution: Dissolved 20 g of Povidone in about 400 mL of purified water in a 600 mL Beaker. Adjusted the pH from 3.59 to pH 11.51 with 10N NaOH. Next heated solution at 75° C. in a constant temperature bath for 30 minutes, to remove residual peroxides. Allowed the solution to cool to room temperature and adjusted the pH from 9.87 to pH 7.01 with 2N HCl. Transferred the solution to a 500 mL volumetric flask using purified water. Diluted to volume with purified water.

Preparation of Stock HPMC Solution: Dissolved 2.5 g of HPMC in about 100 mL of purified water in a 150 mL Beaker. Transferred to a 200 mL volumetric flask using purified water. Diluted to volume with purified water.

Preparation of Stock CMC Solution: Dissolved 2.5 g of CMC in about 100 mL of purified water in a 150 mL Beaker. Transferred to a 200 mL volumetric flask using purified water. Diluted to volume with purified water.

Preparation of Polyvinyl Alcohol Stock Solution: Dissolved 2.5 g of Polyvinyl Alcohol in about 100 mL of purified water in a 150 mL Beaker. Transferred to a 200 mL volumetric flask using purified water. Diluted to volume with purified water.

Preparation of Lab Batches: Lab batches were compounded by first adding the required povidone stock to a 50 mL beaker with a stir bar. If required a modifier was added. 5 mL of stock salt solution was added before taking the pH of the solution. The pH was adjusted to about 7 with 2N HCl. Olopatadine was added and dissolved with mixing. The pH was adjusted again to 7.0 with 1N NaOH. The solution was transferred to a 50 mL volumetric flask with purified water and diluted to volume. After mixing the solution was divided and stored at the specified storage conditions and analyzed.

### HPMC, CMC, and PVA Modifier Evaluation:

The Povidone and modifiers ranged from 1.4% to 1.8% and from 0.1% to 0.5%, respectively during these experiments. A total of 20 formulations were produced for the different modifier evaluations. The formulation viscosities were determined and accelerated stability studies out to 6 days at 40° C. and 80° C. were executed to compare the modified formulations as related to the commercial formulation. The accelerated stability samples at 40° C. did not show a significant difference between the evaluated formulations because the short period of time evaluated and therefore this data is not presented in this report. Only the stability samples at 80° C. showed a difference in the stability trend between modified formulations as related to the commercial Olopatidine Hydrochloride Ophthalmic Solution 0.2% (“control”). The Table 1, Table 2, and Table 3 summarize the major degradant observed, the modifier material used, and study results, respectively.

TABLE 1
Major Degradant Observed

| Relative Retention Time | |
|---|---|
| (RRT) | Degradant |
| 1.14 | Olopatadine N-Oxide |
| 1.20 | Olopatadine Carbaldehyde |

TABLE 2
Modifier Materials Used

| Modifier | Description | CAS # | Viscosity |
|---|---|---|---|
| HPMC | Low | 9004-65-3 | Viscosity of 2% Aqueous Solution |
| | Viscosity | | @ 20° C.: 40-60 mPa * s |
| CMC | Low | 9004-32-4 | Viscosity of 2% Aqueous Solution |
| | Viscosity | | @ 25° C.: 27 mPa * s |
| PVA | Medium | 9002-89-5 | Viscosity of 4% Aqueous Solution |
| | Viscosity | | @ 20° C.: 22.5 mPa * s |

TABLE 3
DoE for New Modifier Evaluations
(Viscosity and Stability @ 80° C. for 144 hours)

| | | | | | N-Oxide | |
|---|---|---|---|---|---|---|
| | | | Vis- | Vis- | 6 | 6 |
| | Povidone | Modifier | cosity | cosity (% | days | days (% |
| Modifier | (%) | (%) | (cPs) | Control) | (%) | Control) |
| Control | 1.80 | 0.00 | 1.34 | 100.00 | 0.68 | 0.70% |
| | 1.80 | 0.00 | 1.34 | 100.00 | 0.72 | (100%) |
| HPMC | 1.40 | 0.10 | 1.55 | 115.72 | 0.59 | 84.29 |
| | 1.80 | 0.10 | 1.62 | 120.75 | 0.73 | 104.29 |
| | 1.40 | 0.50 | 3.19 | 238.36 | 0.62 | 88.57 |
| | 1.80 | 0.50 | 3.53 | 263.52 | 0.72 | 102.86 |
| | 1.60 | 0.30 | 2.33 | 174.21 | 0.61 | 87.14 |
| | 1.60 | 0.30 | 2.33 | 173.58 | 0.55 | 78.57 |
| CMC | 1.40 | 0.10 | 1.54 | 115.09 | 0.41 | 58.57 |
| | 1.80 | 0.10 | 1.61 | 120.13 | 0.54 | 77.14 |
| | 1.40 | 0.50 | 2.76 | 205.66 | 0.36 | 51.43 |
| | 1.80 | 0.50 | 2.96 | 220.75 | 0.40 | 57.14 |
| | 1.60 | 0.30 | 2.09 | 155.97 | 0.43 | 61.43 |
| | 1.60 | 0.30 | 2.07 | 154.72 | 0.42 | 60.00 |
| PVA | 1.40 | 0.10 | 1.31 | 97.48 | 0.50 | 71.43 |
| | 1.80 | 0.10 | 1.42 | 106.29 | 0.66 | 94.29 |
| | 1.40 | 0.50 | 1.63 | 122.01 | 0.37 | 52.86 |
| | 1.80 | 0.50 | 1.77 | 132.08 | 0.50 | 71.43 |
| | 1.60 | 0.30 | 1.51 | 112.58 | 0.47 | 67.14 |
| | 1.60 | 0.30 | 1.52 | 113.21 | 0.55 | 78.57 |

Viscosity Analysis:

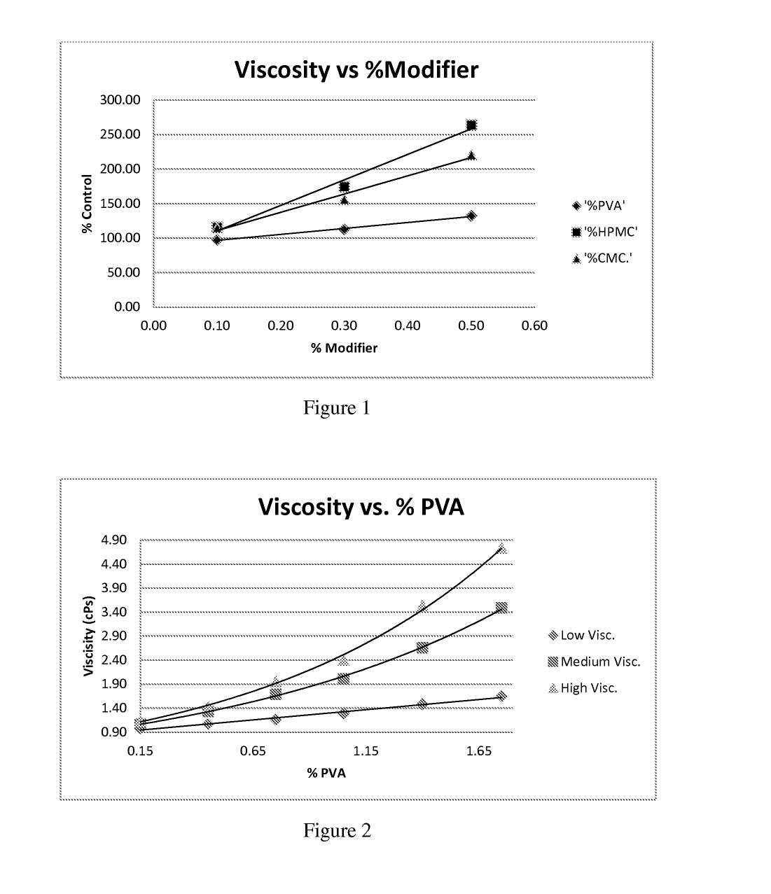

The evaluated modifiers increased the formulation viscosities even at relatively low amounts (0.5%). The HPMC and CMC showed very significant and unacceptable increases at these low amounts while the PVA showed a small increase when compared to the control reference values. FIG. 1 summarizes the viscosity trending reported as percent modifier versus % Control.

A formulation using PVA as a modifier instead of povidone potentially could be manufactured, with an acceptable viscosity (within about 1-2 cPs).

Stability Trends at 80° C.:

The major degradant observed at the 6 days was the Olopatadine N-oxide and it was used for formulation stabilities evaluations. Slightly negative reductions in degradant levels were observed for all formulations containing modifiers, indicating that stable formulations were obtainable. The level of degradant appeared to be directly related to the amount of Povidone present in the formulation. It is believed that this degradation product is produced via oxidation and is related to the residues of peroxides present in the Povidone.

Summary of Design of Experiment (DoE) results:

HPMC: The % of Olopatadine N-oxide ranged from 78.57% to 104.29% related to the control. The % Povidone showed a negative impact in the formulations stabilities. The % HPMC showed a positive impact in the formulations stabilities producing improved stabilities for these formulations when compared with the control. No interaction between the % Povidone and % HPMC was observed during this study.

CMC: The % of Olopatadine N-oxide ranged from 51.43% to 77.14% relative to the control. The % Povidone showed a negative impact in the formulations stabilities. The % CMC showed a very positive impact in the formulations stabilities producing improved stabilities for these formulations when compared with the control. No interaction between the % Povidone and % CMC was observed during this study.

PVA: The % of Olopatadine N-oxide ranged from 52.86% to 94.29% related to the control. The % Povidone showed a negative impact in the formulations stabilities. The % PVA showed a very positive impact in the formulations stabilities producing improved stabilities for these formulations when compared with the control. No interaction between the % Povidone and % PVA was observed during this study.

PVA Modifier Selection:

Based on its minimum impact for the formulation viscosity and it's very positive effect in the formulation stabilities, PVA was further tested. A goal during these studies was to reduce the povidone content, to evaluate the effect of replacing as much as possible of the povidone with PVA. The medium viscosity PVA was used during this study. The experiment described in Table 4 was carried out. During this experiment, the % Povidone was reduced at the same time that an equivalent amount of the % PVA is increased until all of the Povidone was replaced with PVA. For all formulations the total amount of modifiers (Povidone and PVA) was 1.80%. The results for viscosity and formulation stability at 80° C. during four weeks can be observed in Table 4.

TABLE 4
New Olopatadine Formulation Development using PVA

| | | Viscosity | | N-oxide | | Carbaldehyde | |
|---|---|---|---|---|---|---|---|
| | | Vis- | % | 4 | % | 4 | % |
| Povidone | PVA | cosity | Control | Weeks | Control | Weeks | Control |
| (%) | (%) | (cP) | (%) | (%) | (%) | (%) | (%) |
| 1.80 | 0.00 | 1.34 | 100.00 | 1.55 | 100.00 | 0.56 | 100.00 |
| (Control) | | | | | | | |
| 1.20 | 0.60 | 1.72 | 128.21 | 0.54 | 34.84 | 0.35 | 62.5 |
| 0.90 | 0.90 | 1.96 | 146.15 | 0.33 | 21.29 | 0.26 | 46.4 |
| 0.60 | 1.20 | 2.27 | 169.23 | 0.17 | 10.97 | 0.18 | 32.1 |
| 0.00 | 1.80 | 3.08 | 229.49 | 0.00 | 0.00 | 0.00 | 0.00 |

A positive viscosity trend due to increased medium viscosity PVA content in the formulations was observed. However this experiment showed the feasibility of obtaining Olopatadine formulations using PVA with viscosities in the range of 1-2 cPs.

The two major degradation products observed were evaluated against their observed level in the control: Olopatadine N-Oxide and Carbaldehyde. The observed levels of both degradation products decrease when the amounts of Povidone present in the formulations were reduced. The most important result obtained during this experiment was the total absence of both degradation products when the Povidone was totally replaced by PVA demonstrating the superior stability of the formulations containing PVA.

Minimum Amount of PVA:

An experiment was carried out to determine the minimum amount of PVA needed to obtain the total dissolution of Olopatadine in the formulation. The minimum amount of PVA determined to be effective in solubilizing the drug substance during formulation and which prevented crystal growth in the drug product was 0.5%. Concentrations of such PVA below 0.5% are insufficient to permit dissolution of Olopatadine concentrations of about 2.0 mg/mL. Concentrations at or lower than 0.5% were susceptible to the drug substance starting to dissolve but then precipitating back out of solution during formulation. Once the precipitate formed it required significantly more than 0.5% PVA to re-solubilize the precipitate.

Development of Olopatadine Solutions Using Different Grades of PVA:

Samples of low, medium, and high viscosities PVA were obtained from Sigma-Aldrich, see Table 5 for details.

TABLE 5
Three Grades of PVA

| Description | CAS # | Viscosity |
|---|---|---|
| Low Viscosity | 9002-89-5 | Viscosity of 4% Aqueous Solution |
| | | @ 20° C.: 4.2 mPa * s |
| Medium Viscosity | | Viscosity of 4% Aqueous Solution |
| | | @ 20° C.: 23.4 mPa * s |
| High Viscosity | | Viscosity of 4% Aqueous Solution |
| | | @ 20° C.: 48.5 mPa * s |

The first experiment evaluated the viscosity curves for placebos produced with different grades of PVA ranging from 0.15% to 1.75%. Placebo was used to make possible the low PVA content evaluation and because the Olopatidine does not significantly affect the final formulation viscosities. The placebo formulation is summarized in Table 6.

TABLE 6
Summary of Placebo Formulations

| Component | Amount | |
|---|---|---|
| Polyvinyl Alcohol | 0.15-1.75% | |
| EDTA (anhydrous) | 0.1 | mg/mL |
| Disodium Phosphate (anhydrous) | 4.8-5.2 | mg/mL |
| Sodium Chloride | 5.8-6.2 | mg/mL |

| | |
|---|---|
| pH | 6.8-7.2 |

The results obtained during this experiment are summarized in Table 7 and FIG. 2. The results confirmed that it is possible to obtain Olopatadine formulations using individual grades of PVA or the combination of them with viscosities in the range of 1-2 cPs. In general, for the Olopatadine formulations produced with individual grades of PVA in the range from 0.50% to 1.75%, the viscosities will range from about 1.09 cPs to 4.74 cPs. [Refer to Table 8 for formulation summary.]

TABLE 7
Viscosity Curves for Three PVA Grades

| % PVA | Low Viscosity | Medium Viscosity | High Viscosity |
|---|---|---|---|
| 0.15 | 0.98 | 1.06 | 1.14 |
| 0.45 | 1.07 | 1.33 | 1.43 |
| 0.75 | 1.16 | 1.69 | 1.95 |
| 1.05 | 1.29 | 2.01 | 2.40 |
| 1.40 | 1.48 | 2.66 | 3.54 |
| 1.75 | 1.65 | 3.49 | 4.74 |

TABLE 8
Summary of Olopatadine Formulations

| Component | Amount | |
|---|---|---|
| Olopatadine | 1.6-2.3 | mg/mL |
| Olopatadine Hydrochloride | 1.8-2.5 | mg/mL |
| Polyvinyl Alcohol | 0.50-1.75% | |
| EDTA (anhydrous) | 0.1 | mg/mL |
| Disodium Phosphate (anhydrous) | 4.8-5.2 | mg/mL |
| Sodium Chloride | 5.8-6.2 | mg/mL |
| pH | 6.8-7.2 | |
| Viscosity | 1.09-4.74 | cPs |

A second experiment focused on the evaluation of different mixtures of the PVA grades to obtain viscosities similar to the Olopatadine commercial formulation. Based on the results from the previous experiments and the FDA database for pharmaceutical excipients, a Mixture DoE was defined including two levels of total PVA: 0.75% and 1.35%. [Refer to Table 9 for DoE details and viscosity results.] The maximum amount of PVA approved by the FDA for ophthalmic solutions is 1.4%. During this experiment formulations containing Olopatidine Hydrochloride were used to verify the experimental results.

TABLE 9
PVA Mixture DoE

| Low Viscosity | Medium | High Viscosity | Total | |
|---|---|---|---|---|
| PVA | Viscosity PVA | PVA | Amount | Viscosity |
| (%) | (%) | (%) | (%) | (cPs) |
| 0.750 | 0.000 | 0.000 | 0.750 | 1.16 |
| 0.375 | 0.375 | 0.000 | 0.750 | 1.39 |
| 0.375 | 0.000 | 0.375 | 0.750 | 1.51 |
| 0.000 | 0.750 | 0.000 | 0.750 | 1.69 |
| 0.000 | 0.375 | 0.375 | 0.750 | 1.75 |
| 0.000 | 0.000 | 0.750 | 0.750 | 1.95 |
| 0.250 | 0.250 | 0.250 | 0.750 | 1.53 |
| 0.500 | 0.125 | 0.125 | 0.750 | 1.33 |
| 0.125 | 0.500 | 0.125 | 0.750 | 1.57 |
| 0.125 | 0.125 | 0.500 | 0.750 | 1.67 |
| 1.350 | 0.000 | 0.000 | 1.350 | 1.45 |
| 0.675 | 0.675 | 0.000 | 1.350 | 1.93 |
| 0.675 | 0.000 | 0.675 | 1.350 | 2.22 |
| 0.000 | 1.350 | 0.000 | 1.350 | 2.61 |
| 0.000 | 0.675 | 0.675 | 1.350 | 2.95 |
| 0.000 | 0.000 | 1.350 | 1.350 | 3.32 |
| 0.450 | 0.450 | 0.450 | 1.350 | 2.26 |
| 0.900 | 0.225 | 0.225 | 1.350 | 1.81 |
| 0.225 | 0.900 | 0.225 | 1.350 | 2.34 |
| 0.225 | 0.225 | 0.900 | 1.350 | 2.69 |

The Mixture DoE results were processed for both PVA levels: 0.75% and 1.35%. From this study it was concluded that useful Olopatadine formulations can be obtained using individual grades of PVA (or a combination of grades) in amounts between 0.75% and 1.35% to produce viscosities in the range of 1-2 cPs. In general, for the Olopatadine formulations produced with individual grades of PVA in the range from 0.75% to 1.35%, the viscosities will range from 1.16 cPs to 3.32 cPs. [Refer to Table 10 for formulation summary.] The stabilities of these solutions were demonstrated to be similar to the above discussed formulations containing only PVA.

TABLE 10
Summary of Olopatidine Formulations

| Component | Amount | |
|---|---|---|
| Olopatadine | 1.8-2.2 | mg/mL |
| Olopatadine Hydrochloride | 2.0-2.4 | mg/mL |
| Polyvinyl Alcohol | 0.75-1.35% | |
| EDTA (anhydrous) | 0.1 | mg/mL |
| Disodium Phosphate (anhydrous) | 4.8-5.2 | mg/mL |
| Sodium Chloride | 5.8-6.2 | mg/mL |
| pH | 6.8-7.2 | |
| Viscosity | 1.16-3.32 | cPs |

From the data obtained during formulation development, a manufacturing process was derived as follows:
1. Add 90% of water to vessel and heat to 90° C. (Note: polymer is easily soluble at 90 to 98° C.)
2. With high mixing add PVA (individual or mixture of grades) and keep mixing until fully dissolved.
3. Allow solution to cool (35° C.) with mixing.
4. Add Disodium Phosphate, Sodium Chloride and EDTA; mix until dissolved.
5. Ensure solution has cooled (˜25° C.) before next step.
6. Add Olopatadine salt, mix until dissolved.
7. Take pH at (23-25° C.), adjust pH to 7.0±0.2 with HCl.
8. QS to final amount with water.
9. Allow to mix.
10. Filter sterilize the solution.

The organic modifier (PVA) when used alone, can be at a concentration range of above 0.50% and below 1.80%, and in some embodiments between 0.75% and 1.35%. The formulations have similar viscosity, pH, and osmolality versus the commercial Olopatidine Hydrochloride Ophthalmic solution 0.2%. The new formulation showed superior stability than the control during the forced degradation studies. The efficiency of formulation manufacture was improved by eliminating the necessity of the peroxide removal step.

As used herein, when a range is said to be “between” two values, it is meant to include the limits of the range. In other words, between 0.50 and 1.75% includes 0.75 and 1.35%. DoE means design of experiment. “Consisting essentially of menas lacking amounts of polymers and/or nonpolymers that would alter viscosity outside the ranges disclosed as acceptable herein or materially affect stability.

We claim:

1. A pharmaceutical composition comprising an aqueous solution of 11-(3 dimethylaminopropylidene)-6,11-dihydrodibenz(b,e) oxepin-2-acetic acid or a pharmaceutically acceptable salt thereof, and polyvinyl alcohol at a concentration of greater than 0.50% w/w and less than 1.75% w/w, wherein the pH is between 5.0 and 8.0, and the osmolality is between 260 and 340 mOsm/kg.

2. The pharmaceutical composition of claim 1, wherein the 11-(3 dimethylamino)propylidene]-6-11-dihydrodibenz[b,e]oxepin-2-acetic acid is 11-([Z]-3 dimethylaminopropylidene)-6- 11 dihydrodibenz(b,e) oxepin-2-acetic acid.

3. The pharmaceutical composition of claim 1, wherein the 11-(3 dimethylaminopropylidene)-6,11-dihydrodibenz(b,e) oxepin-2-acetic acid is present in an amount between 0.5 mg/mL and 3.0 mg/mL.

4. The pharmaceutical composition of claim 1, wherein the 11-(3 dimethylaminopropylidene)-6,11-dihydrodibenz(b,e) oxepin-2-acetic acid is present in an amount between 1.5 mg/mL and 2.5 mg/mL.

5. The pharmaceutical composition of claim 1, further comprising a chelating agent.

6. The pharmaceutical composition of claim 1, further comprising a buffer.

7. The pharmaceutical composition of claim 6, further wherein the buffer is disodium phosphate and sodium chloride.

8. The pharmaceutical composition of claim 1, wherein the formulation is free of benzalkonium chloride.

9. The pharmaceutical composition of claim 1, wherein the formulation is free of polymeric quaternary ammonium compounds.

10. The pharmaceutical composition of claim 1, wherein the formulation is free of any preservative.

11. The pharmaceutical composition of claim 1, wherein the formulation is free of povidone.

12. The pharmaceutical composition of claim 1, wherein the formulation is free of benzalkonium chloride and free of povidone.

13. The pharmaceutical composition of claim 1, wherein the polyvinyl alcohol is at a concentration of between 0.60 and 1.50% w/w.

14. The pharmaceutical composition of claim 1, wherein the polyvinyl alcohol is at a concentration of between 0.75 and 1.35% w/w.

15. A pharmaceutical composition comprising an aqueous solution containing 11-[(Z)-3-(Dimethylamino)propylidene]-6-11-dihydrodibenz[b,e]oxepin-2-acetic acid present in an amount between 1.5 mg/mL and 2.5 mg/mL, polyvinyl alcohol at a concentration of between 0.50 and 1.75% w/w, disodium phosphate, sodium chloride, and EDTA, and wherein the pH of the composition is between 5.0 and 8.0, and the osmolality is between 260 and 340 mOsm/kg.

16. The pharmaceutical composition of claim 15, wherein the pharmaceutical composition is free of benzalkonium chloride and free of povidone.

17. The pharmaceutical composition of claim 16, wherein the pharmaceutical composition is free of any preservative.

18. A method of treating an allergic condition comprising administering topically to the eye a pharmaceutical composition of claim 1.

19. The pharmaceutical composition of claim 1, wherein the pH is between 6.8 and 7.2.

20. The pharmaceutical composition of claim 15, wherein the pH is between 6.8 and 7.2.
